(19) United States
(12) Patent Application Publication
(10) Pub. No.: US 2016/0256451 A1
(43) Pub. Date: Sep. 8, 2016

(54) DOSAGE OF NALOXONE

(71) Applicant: Develco Pharma Schweiz AG, Pratteln (CH)

(72) Inventors: Helene Rey, Rosenau (FR); Olaf Mundszinger, Efringen-Kirchen (DE); Isabelle Golfier, Bantzenheim (FR); Silvia Jakob, Bernau (DE); Oliver Rusch, Basel (CH)

(21) Appl. No.: 14/828,028

(22) Filed: Aug. 17, 2015

(30) Foreign Application Priority Data
Mar. 6, 2015 (EP) 15170573.8

(51) Int. Cl. A61K 31/485

(52) U.S. Cl. CPC A61K 31/485

(57) ABSTRACT

The present invention relates to a pharmaceutical composition comprising a μ-receptor antagonist as an active substance, wherein the antagonist is prepared in an extended release formulation. The composition is intended for the treatment of patients with opioid-induced constipation, wherein the dosage of the μ-receptor agonist is independent of the opioid dosage. The preferred dosage of the μ-receptor antagonist of the composition is equivalent to a daily dosage of 20-70 mg naloxone.

### BACKGROUND

The present invention relates to a composition comprising as active substance a μ-receptor antagonist, or a pharmaceutically acceptable salt thereof, for use in the treatment of patients with opioid induced constipation, wherein the dosage of the μ-receptor antagonist is independent of the opioid dosage and wherein the active substance is released from the composition in a prolonged manner.

Constipation is a major side effect of opioid analgesics administration. It is one of the most common side effects and is particularly predominant in long-term opioid administration therapies, occurring in approximately 85% of patients. In contrast to other opioid-induced side effects, opioid-induced constipation is a chronic phenomenon, the intensity of which does not decrease over the course of the treatment. The effect of the opioids on the gut mobility is probably due to binding of the opioids to the opioid receptors of the gastrointestinal tract, which are present there at a relatively high density.

The aim of the therapy proposed here is to neutralize this peripheral side effect of opioids because opioid-induced constipation can be uncomfortable and very painful, and often leads to the discontinuation of the opioid-based therapy, and thus endangers the success of the treatment with the opioids. Since it can be assumed that the opioid-induced constipation is caused directly and locally over the entire intestine through binding to the μ-receptor, this side effect should be eliminated through the use of μ-receptor antagonists. However, the use of μ-receptor antagonists only makes sense if the antagonistic effect is limited to the intestine and does not cancel the main analgesic effect.

Naloxone is a suitable μ-receptor antagonist for the treatment of opioid-induced constipation. Naloxone is rapidly and completely absorbed after oral administration and because the substance is subject to extensive first-pass metabolism, only small amounts of unmetabolised naloxone are available to the system. The vast majority of the applied substance is found in blood in the form of inactive or only mildly active metabolites such as naloxone-3-glucuronide or beta-6-naloxol. In suitable doses, naloxone is an ideal candidate for remedying opioid-induced constipation: in the intestine it is present as an active substance and can thus counter the paralysing effect of the opioid on the gastrointestinal tract, while after absorption it is largely metabolised during the first passage in the liver, and thereby becomes inactive. The analgesic effect of the opioids is thus not affected.

Since the paralysis does not only affect the duodenum and the upper part of the small intestine, but the entire gastrointestinal tract, the opioid-induced constipation cannot be treated successfully with a composition that releases the opioid receptor antagonist rapidly. WO 2011/117306 discloses a two-layer tablet, which in one layer contains an opioid agonist, and in another layer an opioid antagonist, wherein the tablet quickly releases both active substances. The advantage of this double-layer is to suppress the side effects of the opioid agonist, but it does not focus on suppression of the opioid-induced constipation.

The combined preparation Targin® is available on the market and comprises a mixture of the opioid agonist oxycodone in the form of a hydrochloric salt, and the opioid antagonist naloxone also in the form of a hydrochloric salt. In this preparation, the active substances are released in a prolonged manner. It is therefore suitable for the parallel treatment of pain and opioid-induced constipation. However, this monolithic formulation has the disadvantage that the release rates of the two active substances are fixed. Individualised treatments are therefore difficult to optimise.

In addition, infusion solutions available on the market for the treatment of opioid poisoning are only naloxone combined preparations, in which naloxone and the opiate are present in a fixed proportion to each other. However, for the treatment of opioid-induced constipation, it would be desirable to have single agent preparations, since this would allow administering μ-receptor antagonist both independently of the nature of the opiate and in variable doses. The desired quantity of μ-receptor antagonist could therefore be applied, which would lead to an optimal treatment. Naloxone single agent preparations are described in the patent literature, such as in WO 98/25613 A2. However, the release of naloxone from these compositions is dependent on the ambient pH in the gastrointestinal tract. A uniform application of naloxone or other μ-receptor antagonists to the entire gastrointestinal tract, and therefore an optimal treatment, are thus not possible with such products.

### BRIEF DESCRIPTION OF THE DRAWINGS

FIG. 1 shows the release profile of naloxone from the composition according to the invention; (x) example 1.

### DESCRIPTION OF THE INVENTION

The present invention addresses those problems by a composition comprising a μ-receptor antagonist, or a pharmaceutically acceptable salt thereof, for use in the treatment of patients with opioid induced constipation,
(i) wherein the antagonist is prepared in an extended release formulation;
(ii) wherein the patients are receiving a continuous opioid treatment; and
(iii) wherein the dosage of the μ-receptor antagonist is independent of the opioid dosage and equivalent to a daily dosage of 20 to 70 mg of naloxone.

The feature “the antagonist is prepared in an extended release formulation” means that the composition comprising a μ-receptor antagonist releases the μ-receptor antagonist in a prolonged manner. A prolonged manner refers to the release of the antagonist over a 2, 4, 6, 8, 10, 12, 14, 16, 18, 20, 22, 24, 36, 48 hour period of time. In some instances it can be longer than 48 hours.

The phrase “receiving an opiate treatment” refers to a patient receiving at least one opiate treatment prior to or at the same time as the opioid receptor antagonist. In some instances, receiving an opiate treatment means more than one opiate treatment is received by the patient. For example, at least one opiate treatment can be prior to administration of the opioid receptor antagonist and at least one opiate treatment can be after administration of the opioid receptor antagonist. In some instances, the opioid treatment can be continuous.

In a preferred embodiment, the daily dosage of μ-receptor antagonist is equivalent to a daily dosage of naloxone of 20-60 mg, more preferably of 22-55 mg and most preferably of 24-48 mg. In a particular embodiment daily dosage of the μ-receptor antagonist is equivalent to a daily dosage of naloxone of 24, 36 or 48 mg.

An amount of an opioid or of an opioid antagonist is to be understood as the indicated amount ±10%, ±8%, ±5%, ±3%, or more preferably ±1%.

In a further embodiment, the opioid μ-receptor antagonist is selected from the group comprising naloxone, methylnaloxone, methylnaltrexone, naltrexone, pharmaceutically acceptable derivatives, in particular sulfonates or esters thereof or pharmaceutically acceptable salts thereof. Most preferably, the opioid μ-receptor antagonist is naloxone.

The objective of the present invention is to provide a pharmaceutical composition comprising a μ-receptor antagonist as an active substance, wherein the antagonist is prepared in an extended release formulation, wherein the patients are receiving a continuous opioid treatment, wherein the dosage of the μ-receptor antagonist is independent of the opioid dosage, for the treatment of opioid-induced constipation.

This objective is achieved by a solid oral pharmaceutical composition comprising a μ-receptor antagonist as an active substance, wherein the composition releases the active substance in a prolonged manner, and the dosage of the μ-receptor antagonist is equivalent to a daily dosage of 20-70 mg naloxone, in a preferred embodiment, the daily dosage of the μ-receptor antagonist is equivalent to a daily dosage of naloxone of 20 to 60 mg, more preferably of 22 to 55 mg and most preferably of 24 to 48 mg. In a particular embodiment daily dosage of the μ-receptor antagonist is equivalent to a daily dosage of naloxone of 24, 36 or 48 mg.

It was observed that the composition according to the invention, with its release profile, was suitable for an administration period of at least twelve-hours for the treatment of opioid-induced constipation. Accordingly it possesses a relatively high level of patient compliance.

It was furthermore observed that a dosage of the μ-receptor antagonist in the composition according to the invention independent of the opioid dosage is effective for use in the treatment of constipation, the dosage of the μ-receptor antagonist being within the range of an amount equivalent to 20-70 mg of naloxone.

The opioid-induced constipation which can be treated by the composition according to the invention can be caused by any opioid analgesic or opioid analgesic analogue, or by any of their salts or mixtures. Examples of such analgesics are the following: alfentanil, allylprodine, alphaprodine, anileridine, benzylmorphine, bezitramide, buprenorphine, butorphanol, clonitazene, codeine, besomorphine, dextromoramide, dezocine, diampromide, diamorphone, dihydrocodeine, dihydromorphine, dimenoxadol, Dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphane, lofentanil, meperidine, meptazinol, metazocine, methadone, metopone, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphane, phenazocine, phenoperidine, piminodine, piritramide, propheptazine, promedol, properidine, propoxyphene, sufentanil, tilidine, and tramadol, wherein hydrocodone, morphine, hydromorphone, oxycodone, buprenorphine, codeine, fentanyl, levorphanol, meperidine, methadone, levomethadone, and dextromethadone are particularly preferred according to the invention.

The in vitro release rate of an active ingredient from a composition is determined using the paddle stirrer apparatus (apparatus 2) with the paddle stirrer method according to Ph. Eur. (European Pharmacopoeia, 7th edition, 3rd supplement, 2.9.3 “Dissolution test for solid dosage forms”, pages 3797-3803) at 75 rpm in 500 ml 0.1 N hydrochloric acid at 37° C. The amount of released active substance is preferably determined by UV-detection at 220 nm.

In a preferred embodiment of the invention, the composition has an in vitro release rate of the active substance of 0% to 50% in 2 h, of 5% to 95% in 4 h, of 20% to 90% in 10 h, of more than 70% in 18 h, and of more than 80% in 24 h.

In a more preferred embodiment of the invention, the composition has an in vitro release rate of the active substance of 0% to 38% in 2 h, of 5% to 55% in 4 h, and of 20% to 75% in 10 h.

According to another preferred embodiment of the invention, the composition has an in vitro release rate of the active substance of 0% to 50% in 1 h, of 10% to 95% in 4 h, of 35% to 100% in 8 h, of 55% to 100% in 12 h, of 70% to 100% in 16 h, and of more than 90% in 24 h.

According to another preferred embodiment of the invention, the composition has an in vitro release rate of the active substance of 0% to 30% in 1 h, of 0% to 40% in 2 h, of 3% to 55% in 4 h, of 10 to 65% in 8 h, of 20% to 75% in 12 h, of 30% to 88% in 16 h, of 50% to 100% in 24 h, and of more than 80% in 36 h.

In a further preferred embodiment of the invention, the composition has an in vitro release rate of the active substance of 10% to 30% in 1 h, of 17% to 37% in 2 h, of 27% to 47% in 4 h, of 40% to 60% in 8 h, of 50% to 70% in 12 h, of 60% to 80% in 16 h, of 80% to 100% in 24 h.

In a particularly preferred embodiment of the invention, the composition releases the μ-receptor antagonist, or the pharmaceutically acceptable salt thereof, independently of the ambient pH of the gastrointestinal tract. This ensures that the entire gastrointestinal tract can be evenly and continuously supplied with the active substance, or an acceptable salt thereof. A further optimisation of the treatment is thereby achieved. The pH-independent release of the active substance from the composition of the invention can be achieved through the choice of suitable pharmaceutical excipients that will be known to the person skilled in the art. Local pH values in the gastrointestinal tract are from about 1.2 (in the stomach), to about 6.8 in the colon.

The release of the active substance from the composition of the invention that is independent from the pH of the gastrointestinal is preferably understood to mean that the similarity factor f2 between a first in vitro release at a pH of 1.2 to 6.8 and a second in vitro release at any other pH of 1.2 to 6.8 is larger or equal to 50.

The similarity factor f2 is determined according to SHAH V. P., TSONG Y., SATHE P., & LIU J. P. (1998), “In vitro dissolution profile comparison-statistics and analysis of the similarity factor, f2”, Pharmaceutical Research, 15, 889-896. Specifically, the similarity factor f2 is calculated by the following formula:

$${f}_{2} = 50 * {log}_{10} ( {[ 1 + \frac{1}{n} {\sum }_{t = 1}^{n} {( {R}_{t} - {T}_{t} )}^{2} ]}^{0.5} * 100 )$$

In this equation, Rt and Tt represent the released quantities of active substance at time point t at the first and second pH. n is the number of time points. The f2 factor is determined under the following conditions: a) the minimal number of time points for one release is 3 (time point 0 is excluded); b) the time points for the first and the second pH should be equal; c) for each time point, and for each pH, the released quantity is indicated as the mean value of 12 measurements; d) no more than one mean value measured above a release of 85% can be taken into account for the calculation; e) the relative standard deviation or coefficient of variation of the release at a given pH should be smaller than 20% for the first time point and smaller than 10% for the second, and every subsequent time point.

In a further preferred embodiment of the invention, the composition comprises a matrix, which releases the active ingredient in a prolonged manner. The active substance can be released in a prolonged manner inexpensively, particularly when it is contained in a matrix that prolongs its release.

The composition according to the invention may comprise a matrix, which releases a μ-receptor antagonist in a prolonged manner. The matrix according to the invention is preferably a so-called scaffold matrix, which can be swelling or non-swelling, or can be a so-called eroding matrix. The matrix can also have properties of both scaffold and eroding matrixes.

In the case of a scaffold matrix, the active substance is incorporated into the matrix structure. The active substance is gradually dissolved by the digestive juices from the loaded scaffold matrix during the transport through the gastrointestinal tract. At the end of the process, the matrix scaffold is excreted in more or less unchanged form, or in a swollen form. In contrast, with an eroding matrix, the matrix is degraded, or eroded, which leads to active substance particles being exposed at the surface, and dissolved. The release rate therefore depends on the matrix degradation or erosion rate.

For the purpose of forming a largely stable scaffold matrix with an appropriate active substance release rate, a further preferred embodiment of the invention is a composition with a matrix that comprises one or several water-insoluble matrix-forming agents.

Another embodiment of the invention is a composition with a matrix that comprises one or several water-soluble matrix-forming agents.

According to a further preferred embodiment of the invention, the matrix of the composition is water-insoluble.

In an alternative embodiment of the invention, the matrix of the composition is water-soluble.

In another preferred embodiment of the invention, the matrix of the composition comprises one or several matrix-forming agents selected from the group consisting of cellulose esters, polyethylene oxide, polyvinylpyrrolidone/polyvinyl acetate mixtures, methacrylate-acrylate copolymers, waxes, fats such as glycerol esters, and fatty alcohols. The substance classes mentioned here are particularly suitable as matrix-forming agents for the composition of the invention. However, particularly preferred is the use of a mixture of polyvinyl acetate and polyvinylpyrrolidone, and/or a glycerol dibehenic acid ester as matrix-forming agent.

In a further preferred embodiment of the invention, the composition is free of film-coated, μ-receptor antagonist-containing particles, wherein the coating causes the prolonged release of the μ-receptor antagonist.

According to a further preferred embodiment of the invention, the composition can be formed by direct compression, since this is particularly inexpensive.

According to another preferred embodiment of the invention, the composition is in the form of a tablet, capsule, granule, a micro tablet, extruded particles or granules compressed into a tablet.

In a further preferred embodiment of the invention, the composition is designed as a once-a-day formulation, or a twice-a-day formulation.

As a μ-receptor antagonist, Naloxone, or a pharmaceutically acceptable salt thereof, is the preferred active substance of the composition according to the invention, wherein naloxone hydrochloride is particularly preferred due to its solubility and stability. One or several additional active substances can be present in the composition.

Regarding the composition which is particularly suited for a twice-a-day administration, the present invention further relates to a solid oral pharmaceutical composition comprising a μ-receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 5% to 50% in 1 h, of 10% to 75% in 2 h, of 20% to 95% in 4 h, of 40% to 100% in 8 h, of more than 50% in 12 h, of more than 70% in 18 h, and of more than 80% in 24 h.

Regarding the composition which is particularly suited for a twice-a-day administration, the present invention further relates to a solid oral pharmaceutical composition comprising a μ-receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 20% to 50% in 1 h, of 40% to 75% in 2 h, of 60% to 95% in 4 h, of 80% to 100% in 8 h, and of 90% to 100% in 12 h.

Regarding the composition which is particularly suited for a once-a-day administration, the present invention further relates to a solid oral pharmaceutical composition comprising a μ-receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 50% in 1 h, of 0% to 75% in 2 h, of 10% to 95% in 4 h, of 35% to 100% in 8 h, of 55% to 100% in 12 h, of 70% to 100% in 16 h and of more than 90% in 24 h.

Regarding the composition which is particularly suited for a once-a-day administration, the present invention further relates to a solid oral pharmaceutical composition comprising a μ-receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 30% in 1 h, of 0% to 40% in 2 h, of 3% to 55% in 4 h, of 10% to 60% in 8 h, of 20% to 75% in 12 h, of 30% to 88% in 16 h, of 50% to 100% in 24 h, and of more than 80% in 36 h.

Regarding the composition which is particularly suited for a once-a-day administration, the present invention further relates to a solid oral pharmaceutical composition comprising a μ-receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 10% to 30% in 1 h, of 17% to 37% in 2 h, of 27% to 47% in 4 h, of 40% to 60% in 8 h, of 50% to 70% in 12 h, of 60% to 80% in 16 h, and of 80% to 100% in 24 h.

In accordance with good patient compliance, a further preferred embodiment of the invention is a composition, wherein the composition is preferably a tablet or a capsule, which has an in vitro release rate of the active substance, measured using the paddle stirrer method according to Ph. Eur. at 75 rpm in 500 ml 0.1 N hydrochloric acid at 37° C., of 0% to 75% in 2 h, of 3% to 95% in 4 h, of 20% to 100% in 10 h, of 30% to 100% in 16 h, of 50% to 100% in 24 h, and of more than 80% in 36 h.

A further preferred embodiment of the invention is providing a composition that is suitable for the treatment of opioid-induced constipation for at least 12 h, provided that the composition has an in vitro release rate of the active substance of 0% to 50% in 2 h, of 5% to 95% in 4 h, of 20% to 90% in 10 h, of more than 70% in 18 h, and of more than 80% in 24 h.

The release rate is, in accordance with the invention, controlled by adjusting the mass ration of μ-receptor antagonist to matrix-forming agent. In a preferred embodiment, the mass ratio of μ-receptor antagonist to matrix-forming agent is 1:1, more preferably 1:2, more preferably 1:5, more preferably 1:10, more preferably 1:20, even more preferably 1:50, yet more preferably 1:75 and most preferably 1:100.

The composition of the invention is characterised in that through the prolonged release the concentration of μ-receptor antagonist in the plasma is low. Its maximum plasma concentration (Cₘₐₓ) is about 20× lower during the active course compared to a composition without prolonged release, and about 100× lower compared with an intravenously administered composition.

However, the inhibition of the receptors over the active course is better. In addition to providing the constipation prevention effect of the μ-receptor antagonist, the low bioavailability in the system also ensures a reduced likelihood and/or severity of the side effects.

Since the naloxone inhibitory concentrations (IC₅₀) for opioid receptors (μ, δ and κ) are known, the assessment of the risk factor of a tablet can be calculated with the ratio IC₅₀/Cₘₐₓ. With the IC₅₀ of μ receptor, the value of IC₅₀/Cₘₐₓ for a tablet according to the invention with 48 mg of naloxone is 54. In general, the higher the value of IC₅₀/Cₘₐₓ, the lower the risk factor of the tablet according to the invention. Hereafter all values relating to the IC₅₀ are for the μ receptor.

In a preferred embodiment, the composition has an IC₅₀/Cₘₐₓ value of at least 30. In a more preferred embodiment, the composition has an IC₅₀/Cₘₐₓ value of at least 35. In an even more preferred embodiment, the composition has an IC₅₀/Cₘₐₓ value of at least 40. In the most preferred embodiment, the composition has an IC₅₀/Cₘₐₓ value of at least 50.

In a further embodiment, the composition additionally comprises at least one stabilizer, which protects the active substance. In a preferred embodiment, the at least one stabilizer is selected from the list comprising sulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals or other metals, paraben, BHA, BHT, gallates, as well as lower fatty acids, fruit acids, phosphoric acids, sorbic and benzoic acids as well as their salts, esters, derivatives and isomeric compounds, ascorbyl palmitate, lecithins, mono- and polyhydroxylated benzene derivatives, ethylenediaminetetraacetic acid and salts thereof, citraconic acid, cysteine, L-cysteine, conidendrin, diethyl carbonate, methylenedioxyphenols, cephalin, β,β′-dithiopropionic acid, biphenyl and other phenyl derivatives.

In a further embodiment, the composition additionally comprises at least one stabilizer, which protects the matrix. In a preferred embodiment, the at least one stabilizer is selected from the list comprising butylated hydroxytoluol, sulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals and other metals, paraben, BHA, BHT, gallates as well as lower fatty acids, fruit acids, phosphoric acids, sorbic and benzoic acids and their salts, esters, derivatives and isomeric compounds, ascorbyl palmitate, lecithins, mono- and polyhydroxylated benzene derivatives, ethylenediaminetetraacetic acid and their salts, citraconic acid, cysteine, L-cysteine, conidendrin, diethyl carbonate, methylenedioxyphenole, cephalin, β,β′-dithiopropionic acid, biphenyl and other phenyl derivatives.

In a further embodiment, the composition comprises at least one additive, wherein the additive is an emetic or a pungent agent drug. In a preferred embodiment, the composition comprises an additive, wherein this additive is a pungent agent, selected from the group comprising Allii sativi bulb, Asari rhizome cum herba, Calami rhizoma, capsici fructus (capsicum} capsici fructus acer (cayenne pepper), Rhizoma Curcumae Longae, Curcumae xanthorrhizae rhizoma, Galangae rhizoma, Semen Myristicae, Piperis nigri fructus (pepper), Sinapis albae (Erucae) Semen, Sinapis nigrae semen, Zedoariae rhizoma and Zingiberis rhizoma, preferably from the group consisting of capsici fructus (capsicum), capsici fructus acer (cayenne pepper) and Piperis nigri fructus (pepper).

In a preferred embodiment, the composition comprises at least one additive, wherein this additive is an emetic. In a preferred embodiment, the emetic is based on one or several substances from radix ipecacuanha (ipecac). In a preferred embodiment, the emetic is based on the substance emetine, in an alternative embodiment, the emetic is apomorphine.

In a further embodiment, the composition comprises a dye. In a preferred embodiment, the dye is selected from a group comprising red iron oxide, black iron oxide and indigo carmine.

In a further embodiment, the composition additionally comprises at least one non-steroid antirheumatic or an antihistamine.

In an alternative embodiment, the composition additionally comprises at least one water-soluble lubricant. In a preferred embodiment, the composition comprises at least one water-soluble lubricant selected from the group comprising adipic acid, fumaric acid, sodium benzoate and macrogol.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment. Independently of the opioid treatment can refer to the administration of the μ-receptor antagonist in a composition that is formulated separately from the opioid treatment.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the μ-receptor antagonist is naloxone, methylnaloxone, methylnaltrexone, naltexone, or pharmaceutically acceptable salts thereof.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the dosage of the μ-receptor antagonist is equivalent to a daily dosage of 24-48 mg of naloxone.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the composition is designed as a once-a-day formulation. In some instances, the composition is designed as a twice-a-day formulation. For example, the extended release formulation can have a 12-hour or a 24-hour release rate.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the composition releases the μ-receptor antagonist independently of the ambient pH of the gastrointestinal tract. In some instances, the μ-receptor antagonist has an in vitro release rate measured using the paddle stirrer method at 75 rpm in 500 ml 0.1 N hydrochloric acid at 37° C., is 0% to 75% in 2 h, 3% to 95% in 4 h, 20% to 100% in 10 h, 30% to 100% in 16 h, 50% to 100% in 24 h, and of more than 80% in 36 h.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the composition is a solid oral composition.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the μ-receptor antagonist is naloxone.

Disclosed are methods comprising administering a composition comprising an extended release formulation of a μ-receptor antagonist in a dosage equivalent to a daily dosage of 20 mg to 70 mg of naloxone to a subject receiving an opioid treatment, wherein the μ-receptor antagonist can be administered independently of the opioid treatment, wherein the opioid treatment is a continuous opioid treatment. Continuous opioid treatment can refer to the opioid being administered at set times for a period of time. For example, the opioid can be administered every other day for six weeks, or every day for 2 weeks. In some instances, continuous opioid treatment can refer to the opioid being administered non-stop, for example, intraveneously, for a period of time.

Disclosed are methods comprising administering a composition comprising an extended release formulation of a μ-receptor antagonist in a dosage equivalent to a daily dosage of 20 mg to 70 mg of naloxone to a subject receiving an opioid treatment, further comprising administering an opioid to the subject, wherein the μ-receptor antagonist is not naloxone or wherein the opioid is not oxycodone or both. In some instances, the opioid can be present in the composition comprising an extended release formulation of a μ-receptor antagonist.

Disclosed are methods comprising administering a composition comprising an extended release formulation of a μ-receptor antagonist in a dosage equivalent to a daily dosage of 20 mg to 70 mg of naloxone to a subject receiving an opioid treatment further comprising administering an opioid to the subject wherein the opioid: μ-receptor antagonist molar ratio is greater than about 2.1:1. In some instances, the opioid: μ-receptor antagonist molar ratio can be, but is not limited to, greater than about 2.5:1, 3:1, 3.5:1, 4:1, 4.5:1, or 5:1.

Disclosed are methods comprising administering a composition comprising an extended release formulation of a μ-receptor antagonist in a dosage equivalent to a daily dosage of 20 mg to 70 mg of naloxone to a subject receiving an opioid treatment further comprising administering an opioid to the subject wherein the opioid: μ-receptor antagonist molar ratio is less than about 2:1. In some instances, the opioid: μ-receptor antagonist molar ratio can be, but is not limited to, less than about 1.9:1, 1.5:1, 1:1, or 0.5:1.

Disclosed are compositions comprising an opioid and a μ-receptor antagonist, wherein the μ-receptor antagonist is not naloxone.

Disclosed are compositions comprising an opioid and a μ-receptor antagonist, wherein the opioid is not oxycodone.

Disclosed are compositions comprising an opioid and a μ-receptor antagonist, wherein there is greater than about a 2.1:1 molar ratio of opioid to μ-receptor antagonist. In some instances, the opioid: μ-receptor antagonist molar ratio can be, but is not limited to, greater than about 2.5:1, 3:1, 3.5:1, 4:1, 4.5:1, or 5:1

Disclosed are compositions comprising an opioid and a μ-receptor antagonist, wherein there is less than about a 2:1 molar ratio of opioid to μ-receptor antagonist. In some instances, the opioid: μ-receptor antagonist molar ratio can be, but is not limited to, less than about 1.9:1, 1.5:1, 1:1, or 0.5:1.

The following examples serve to illustrate the invention.

### EXAMPLES

### Oral Composition

The following examples are used in conjunction with the drawing to illustrate the invention. It shows:

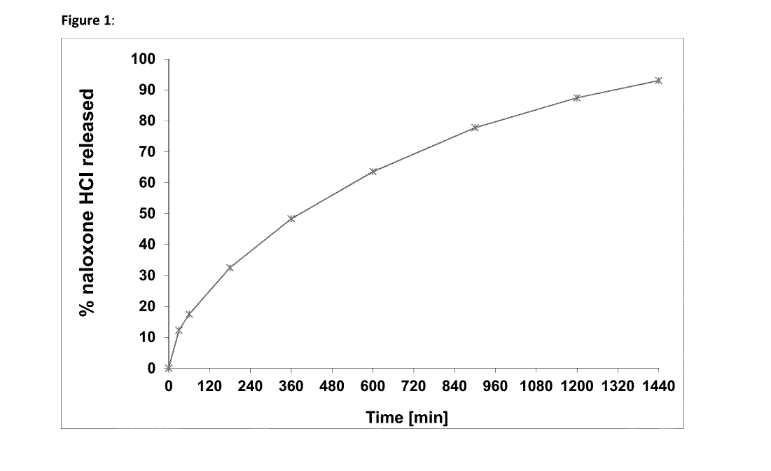

FIG. 1: Release profile of the tablets according to example 1.

### Example 1

Tablets with the following composition were produced:

| | | Weight |
|---|---|---|
| Substance | Function | [mg] |
| Naloxone hydrochloride | Active substance | 48.00 |
| Glycerol dibehenic acid ester | Release retardant | 204.64 |
| Colloidal silicon dioxide | Flow regulator | 19.00 |
| Magnesium stearate | Lubricant | 2.36 |
| | Total weight of the tablet | 274.00 |

The components naloxone hydrochloride and glycerol dibehenic acid ester were sieved and mixed together. First the sieved Colloidal silicon dioxide and then the magnesium stearate were mixed into the resulting mixture. The thus obtained mixture was pressed into a tablet using a conventional tablet pressing tool.

### Example 2

Tablets with the following composition were produced with the same method as in example 1:

| | | Weight |
|---|---|---|
| Substance | Function | [mg] |
| Naloxone hydrochloride | Active substance | 48.00 |
| Kollidon ® SR | Release retardant | 105.00 |
| Vivapur 200 | Filler | 60.65 |
| Glycerol dibehenic acid ester | Release retardant | 95.00 |
| Colloidal silicon dioxide | Flow regulator | 7.47 |
| Magnesium stearate | Lubricant | 1.88 |
| | Total weight of the tablet | 318.00 |

Kollidon® SR consisting of 80 wt.-% polyvinyl acetate, 19 wt.-% povidone, 0.8 wt.-% sodium lauryl sulfate and 0.2 wt.-% Colloidal silicon dioxide.

### Example 3

Coated two-layer tablets with the following composition were produced:

| | | Weight |
|---|---|---|
| Substance | Function | [mg] |
| Naloxone layer | | |
| Naloxone Hydrochloride | Active substance | 48.00 |
| Kollidon ® SR | Release retardant | 204.00 |
| Microcrystalline cellulose | Filler | 84.00 |
| Colloidal silicon dioxide | Flow regulator | 10.00 |
| Magnesium stearate | Lubricant | 2.00 |
| | Total weight of the | 348.00 |
| | naloxone layer | |
| Placebo layer | | |
| Sugar pellets (diameter: 500-600 μm) | Carrier | 80.00 |
| Cellulose | Filler | 242.00 |
| Colloidal silicon dioxide | Flow regulator | 4.00 |
| Magnesium stearate | Lubricant | 4.00 |
| | Total weight of the | 330.00 |
| | placebo layer | |
| | Total weight of the | 678.00 |
| | two-layer tablet core | |
| Opadry ^{®} | Tablet coating | 22.00 |
| | Total weight of the | 700.00 |
| | two-layer tablet | |

The components of the naloxone layer, that is, naloxone hydrochloride, Kollidon® SR, colloidal silicon dioxide and magnesium stearate were sieved and blended together to form a first powdery mixture.

Further, the components of the placebo layer: sugar pellets, microcrystalline cellulose, colloidal silicon dioxide and magnesium stearate were sieved and mixed together to form a second powdery mixture.

The first and the second mixture were pressed with a conventional two-layer tablet press to obtain the two-layer tablet core. The thus obtained two-layer tablet core was coated to obtain the two-layer tablet.

### Release Profile

The in vitro release profile of the tablets according to example 1 was determined using a the paddle stirrer apparatus (apparatus 2) with the paddle stirrer method according to Ph. Eur. (European Pharmacopoeia, 7th edition, 3rd supplement, 2.9.3 “Dissolution test for solid dosage forms”, pages 3797-3803) at 75 rpm in 500 ml 0.1 N hydrochloric acid at 37° C. The amount of released naloxone was determined by UV-detection at 220 nm.

The in vitro release profile of the tablets according to example 1 (x) is shown in FIG. 1.

1. A method comprising:
administering a composition comprising an extended release formulation of a μ-receptor antagonist in a dosage equivalent to a daily dosage of 20 mg to 70 mg of naloxone to a subject receiving an opioid treatment,
wherein the μ-receptor antagonist is administered independently of the opioid treatment.

2. The method of claim 1, wherein the μ-receptor antagonist is naloxone, methylnaloxone, methylnaltrexone, naltexone, or pharmaceutically acceptable salts thereof.

3. The method of claim 1, wherein the dosage of the μ-receptor antagonist is equivalent to a daily dosage of 24-48 mg of naloxone.

4. The method of claim 1, wherein the composition is designed as a once-a-day formulation.

5. The method of claim 1, wherein the composition is designed as a twice-a-day formulation.

6. The method of claim 1, wherein the composition releases the μ-receptor antagonist independently of the ambient pH of the gastrointestinal tract.

7. The method of claim 1, wherein the μ-receptor antagonist has an in vitro release rate measured using the paddle stirrer method at 75 rpm in 500 ml 0.1 N hydrochloric acid at 37° C., is 0% to 75% in 2 h, 3% to 95% in 4 h, 20% to 100% in 10 h, 30% to 100% in 16 h, 50% to 100% in 24 h, and of more than 80% in 36 h.

8. The method of claim 1, wherein the composition is a solid oral composition.

9. The method of claim 2, wherein the μ-receptor antagonist is naloxone.

10. The method of claim 1, wherein the opioid treatment is a continuous opioid treatment.

11. The method of claim 1, further comprising administering an opioid to the subject, wherein the μ-receptor antagonist is not naloxone.

12. The method of claim 1, further comprising administering an opioid to the subject, wherein the opioid is not oxycodone.

13. The method of claim 12, wherein the opioid is present in the composition comprising an extended release formulation of a μ-receptor antagonist.

14. The method of claim 1 further comprising administering an opioid to the subject wherein the opioid: μ-receptor antagonist molar ratio is greater than about 2.1:1.

15. The method of claim 1 further comprising administering an opioid to the subject wherein the opioid: μ-receptor antagonist molar ratio is less than about 2:1.

16. A composition comprising an opioid and a μ-receptor antagonist, wherein the μ-receptor antagonist is not naloxone.

17. A composition comprising an opioid and a μ-receptor antagonist, wherein the opioid is not oxycodone.

18. A composition comprising an opioid and a μ-receptor antagonist, wherein there is greater than about a 2.1:1 molar ratio of opioid to μ-receptor antagonist.

19. A composition comprising an opioid and a μ-receptor antagonist, wherein there is less than about a 2:1 molar ratio of opioid to μ-receptor antagonist.

20. The method of claim 11, wherein the opioid is present in the composition comprising an extended release formulation of a μ-receptor antagonist
